(19) United States
(12) Patent Application Publication
(10) Pub. No.: US 2015/0036102 A1
(43) Pub. Date: Feb. 5, 2015

(54) SLOWING MYOPIA PROGRESSION AND/OR THE TREATMENT OR PREVENTION OF MYOPIA OR A DISEASE OR CONDITION ASSOCIATED WITH MYOPIA

(71) Applicant: Queensland University of Technology, Brisbane, Queensland (AU)

(72) Inventors: Atanu Ghosh, Everton Park (AU); Michael John Collins, Jollys Lookout (AU); Scott Andrew Read, Mitchelton (AU); Brett Ashley Davis, Holland Park (AU)

(73) Assignee: Queensland University of Technology, Brisbane, Queensland (AU)

(21) Appl. No.: 14/380,058

(22) PCT Filed: Feb. 21, 2013
(86) PCT No.: PCT/AU2013/000160

§ 371 Date: Aug. 20, 2014

Related U.S. Application Data
(60) Provisional application No. 61/601,202, filed on Feb. 21, 2012.

(51) Int. Cl. G02C 7/02; G02C 7/06; A61B 3/10; G02C 7/14

(52) U.S. Cl. CPC G02C 7/02; G02C 7/14; G02C 7/06; G02C 7/061; A61B 3/10; G02C 7/027; G02C 2202/24
USPC 351/205; 351/159.58; 351/159.45; 351/159.42; 351/159.79; 351/246; 351/159.74

(57) ABSTRACT

The invention provides an ophthalmic lens comprising one or more oblique prismatic component, wherein the lens slows myopia progression and/or treats or prevents myopia or a disease or condition associated with myopia. The one or more oblique prismatic components may be base-down or base-down and base-in or base up and base-in. The one or more oblique prismatic components may be central and/or in a distance and/or near zone of the ophthalmic lens. The invention also provides an ophthalmic lens comprising a central base-down prism in a distance zone wherein the lens slows myopia progression and/or treats or prevents myopia or a disease or condition associated with myopia. Also provided is an optical device comprising one or more ophthalmic lenses and a method of slowing myopia progression and/or treating or preventing myopia or a disease or condition associated with myopia including using one or more ophthalmic lenses.

### FIELD

THIS INVENTION described herein relates generally to a lens, device and method for slowing myopia progression and/or the treatment or prevention of myopia or a disease or condition associated with myopia. In particular, the invention is directed to a lens and device comprising a vertical or an oblique prism or a central base-down prism and method for slowing myopia progression and/or the treatment or prevention myopia or a disease or condition associated with myopia using the lens or device, although the scope of the invention is not necessarily limited thereto.

### BACKGROUND

Myopia is an important global cause of reduced vision and is one of the priorities for the “Vision 2020” initiative by the World Health Organisation (WHO). The onset of myopia, or short-sightedness, can occur in infants, children or adults. When myopia onset occurs in children the level of myopia can keep increasing until adulthood. The progression of myopia to high levels can have a negative impact on the quality of life and increases the risk of serious eye problems such as glaucoma and retinal degenerations in later life. A variety of factors have been shown to be involved in myopic progression including environmental factors and genetics.

Some studies have shown bifocal spectacles can slightly slow myopic progression, in children. However, improved treatments are required to more effectively slow and/or treat myopia.

### SUMMARY

The present invention is broadly directed to an ophthalmic lens, device and method for slowing myopia progression and/or the treatment and prevention of myopia or a disease or condition associated with myopia. Surprisingly, the inventors have discovered that an ophthalmic lens comprising a vertical or an oblique prism may slow myopia progression and/or treat or prevent myopia or a disease or condition associated with myopia. Of significant advantage, the invention may reduce extra-ocular muscle tension and thereby slow myopic progression and/or treat or prevent myopia or a disease or condition associated with myopia. The present inventors have also provided an ophthalmic lens and device comprising a central base-down prism in a distance zone that may also be able to slow myopia progression and/or treat or prevent myopia or a disease or condition associated with myopia.

In a first aspect there is provided an ophthalmic lens comprising one or more vertical or oblique prismatic components, wherein the lens slows myopia progression and/or treats or prevents myopia or a disease or condition associated with myopia.

In one embodiment of the first aspect the one or more vertical prismatic components may be comprised of base down prism or the one or more oblique prismatic components may be comprised of base-down and base-in prism.

The vertical prismatic components may be comprised of base down prism.

The oblique prismatic components may be comprised of base down and base in prism.

In another embodiment of the first aspect the one or more oblique prismatic components may be comprised of base up and base-in prism.

In one embodiment of the first aspect the one or more vertical or oblique prismatic components may be central.

The central one or more oblique or vertical prismatic components may be central to the direction of the gaze.

In one embodiment of the first aspect the one or more oblique or vertical prismatic components may be in a distance zone and/or a near zone of the ophthalmic lens.

In one embodiment of the first aspect the one or more oblique prismatic components may be oriented at 1 to 45° obliquity to the vertical meridian (90°). In preferred embodiments the obliquity may be 5 to 45, 5 to 35°; 5 to 25° or 5 to 10° to the vertical meridian (90°). The obliquity may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45° to the vertical meridian (90°).

The angle of the obliquity may be constant or variable. The magnitude of the one or more oblique or prismatic components may be constant or variable.

The obliquity and/or power may increase from a distance centre to a near zone centre of the lens.

The obliquity and/or power may each be an independent continuous function.

In an illustrative embodiment the prism dioptric level and obliquity may increase from 1.0Δ and 0°, respectively; to 2.0Δ and 4°, respectively; to 3.0Δ and 9°, respectively; to 4.0Δ and 21°.

In another illustrative embodiment comprising a plurality of base-down and base-in oblique prisms, the oblique prisms may comprise progressive addition of prismatic power from 4Δ to 8Δ.

The magnitude of the prism dioptres of the one or more oblique prismatic components may be 0.1, 0.2, 0.3, 0.4, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 110.0, 11.5, 12.0, 12.5, 13.0, 13.5, 14.0, 14.5, 15.0, 15.5, 16.0, 16.5, 17.0, 17.5, 18.0, 18.5, 19.0, 19.5 or 20.0. In one embodiment the dioptric level is between 0.1 and 20.0, 0.5 and 15 prism dioptres. In another embodiment the dioptric level is between 4 and 12 prism dioptres.

The angle of the obliquity and/or the magnitude of the one or more oblique prismatic components may be a constant or variable function. In a preferred embodiment the angle of the obliquity and/or the magnitude of the one or more oblique prismatic components is a constant function.

The angle of the obliquity of the one or more oblique prismatic components may be variable from the distance centre to the near zone centre.

The magnitude of the one or more oblique prismatic components may be variable from the distance centre to the near zone centre.

In one embodiment of the first aspect the inter-pupillary distance may be 40 to 80 mm. In a preferred embodiment the inter-pupillary distance may be 55 to 70 mm. The inter-pupillary distance may be 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 or 80 mm.

In one embodiment of the first aspect the near inter-pupillary distance may be 35 to 75 mm. In a preferred embodiment the near inter-pupillary distance may be 48 to 68 mm. The near inter-pupillary distance may be 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74 or 75 mm.

In one embodiment of the first aspect the lens may further comprise progressive addition of positive dioptric power and/or or one or more distinct zones of positive addition dioptric power.

The positive dioptic power may be comprised in a single vision power lens, bifocal lens, a trifocal lens or a progressive style lens.

In one embodiment of the first aspect the slowing of myopia progression and/or treatment or prevention of myopia may be over a period of time. The period of time may be weeks, months or years.

In one embodiment of the first aspect one or more oblique prismatic components comprises or is a central base-down and base-in oblique prism.

In one embodiment of the first aspect the one or more oblique prismatic components comprises or is a plurality of base-up and base-in oblique prismatic components.

In one embodiment of the first aspect the ophthalmic lens comprises progressive addition and the one or more oblique prismatic components comprises or is a plurality of base-down and base-in oblique prismatic components.

In one embodiment of the first aspect the ophthalmic lens reduces extra-ocular muscle tension or forces. The tension and forces may be reduced during activities that require angled gaze. The angled gaze may be down and in or up and in.

In a second aspect there is provided an ophthalmic lens comprising a central base-down prism in a distance zone wherein the lens slows myopia progression and/or treats or prevents myopia or a disease or condition associated with myopia.

In one embodiment of the second aspect the slowing of myopia progression and/or treatment or prevention of myopia may be over a period of time. The period of time may be weeks, months or years.

In a third aspect there is provided an optical device comprising one or more ophthalmic lenses, the one or more lenses each comprising the lens of the first or second embodiment.

The optical device may comprise a distance, intermediate or near single vision optical device.

In a preferred embodiment of the third aspect the optical device comprises spectacles.

In another embodiment of the third aspect the optical device comprises one or more variable ophthalmic lens wherein the one or more oblique prismatic components or the central base-down prism in a distance zone is altered with changing gaze angle.

The gaze angle may be tracked with a camera.

According to any above aspect the slowing of myopia progression and/or treatment or prevention of myopia or a disease or condition associated with myopia may comprise slowing or stopping an increase in axial length and/or reducing extra-ocular muscle tension or forces.

In a fourth aspect the invention provides a method of slowing myopia progression and/or treating or preventing myopia or a disease or condition associated with myopia including using one or more ophthalmic lenses according to the first or second aspect or an optical device according to the third aspect to thereby slow myopia progression and/or treat or prevent myopia.

In a sixth aspect the invention provides a method for slowing myopia progression and/or treating or preventing myopia or a disease or condition associated with myopia including identifying optical or eye length changes associated with extra-ocular muscle tension or forces and correcting or inhibiting the optical or eye length changes with an ophthalmic lens according to the first or second embodiment or the optical device according to the third embodiment.

In one embodiment of the sixth aspect the optical or eye length changes may comprise changes due to extra-ocular muscle tension related to angle of gaze.

The angle of gaze may be up and in or down and in.

In a seventh aspect the invention provides a method for measuring a change in optical correction required with gaze angle comprising:

measuring a first axial measurement of an eye at a first gaze angle;

measuring two or more further axial measurements of the eye at respective additional and different gaze angles; and

analysing the first and further axial measurements to determine the change in optical correction of the eye required.

The method of the seventh aspect may further comprise applying the determined change in optical correction to design a lens.

In an eighth aspect the invention provides an ophthalmic lens designed according to the seventh aspect.

In a ninth aspect the invention provides a pair of spectacles comprising one or two ophthalmic lenses of the eighth aspect.

The one or two ophthalmic lenses may comprise a variable lens.

The spectacles of the ninth aspect may be for intermediate and/or near tasks.

The intermediate task may be computer work.

The near task may be reading handheld material such as a book, e-book, tablet computer, phone, electronic device, magazine or newspaper.

In a tenth aspect the invention provides a method of designing a lens including measuring changes in axial length associated with gaze angle and eliminating or correcting these changes with a lens comprising one or more oblique or vertical prismatic components.

The measuring changes in axial length according to the eighth aspect may comprise the method of the seventh aspect.

In an eleventh aspect the invention provides a device for measuring a change in optical correction required with gaze angle comprising:

a biometer for measuring a first axial measurement of an eye at a first gaze angle and for measuring two or more further axial measurements of the eye at respective additional and different gaze angles.

The device of the eleventh aspect may comprise a tilting device for altering the angle of the biometer relative to the eye.

The tilting device may comprise a mechanical device or an optical device.

The device of the eleventh aspect may comprise a head tracker to track a subject's head position during the first and further measurements.

The head tracker may track head position in 3-axes comprising roll, pitch and yaw.

According to any above aspect the disease or condition associated with myopia may comprise one or more of cataract, glaucoma, chorioretinal disease, retinal detachment, lacquer crack, chorioretinal atrophy, lattice degeneration and/or posterior vitreous detachment.

As used herein, except where the context requires otherwise, the term “comprise” and variations of the term, such as “comprising”, “comprises” and “comprised”, are not intended to exclude further additives, components, integers or steps.

### BRIEF DESCRIPTION OF THE DRAWINGS

In order that the present invention may be readily understood and put into practical effect, reference will now be made to the accompanying illustrations, wherein like reference numerals refer to like features and wherein:

FIG. 1 shows variable power and angle lens according to one embodiment of the invention.

FIGS. 2A, 2B, 3 and 4 show various embodiments of a lens according to the invention.

FIGS. 5A and 5B show a schematic diagram and digital photo of one embodiment of a measuring set-up suitable for use in the invention.

FIG. 6A shows one embodiment of a tilted measuring device according to the invention.

FIGS. 6B-6D shows a measurement during a control condition and with changes in eye angle and head angle according to one embodiment of the invention.

FIG. 7 shows a series of bar graphs summarising results according to one embodiment of the invention.

FIG. 8 shows a line graph showing results according to embodiments of the invention.

FIG. 9 shows a bar graph showing results according to one embodiment of the invention.

FIG. 10 shows another view of a tilted measuring device according to the invention.

FIG. 10A shows a subject performing a near task with 25° convergence and 2D accommodation in 10° downward gaze.

FIG. 11 is a summary of different test conditions according to one embodiment of the invention.

FIG. 12 is a schematic diagram of various test conditions according to one embodiment of the invention.

FIG. 13A (top) shows a front view of one embodiment of a head tracker according to the invention.

FIG. 13B (bottom) shows the custom built head tracker shown in FIG. 13A mounted in use to a user's head.

FIG. 14 illustrates the group mean changes of axial length (AXL) (n=7) in right eye during various natural visual tasks (i.e. watching TV, computer work and reading books) with a combined effect of accommodation, convergence and gaze angle. Positive angles correspond to convergence and negative angles correspond to divergence.

### DETAILED DESCRIPTION

The inventors have produced an ophthalmic lens and device that may advantageously prevent myopia progression and/or treat or prevent myopia or a disease or condition associated with myopia. Surprisingly, as exemplified herein the inventors have discovered that provision of an ophthalmic lens comprising an oblique or vertical prism and/or a central base-down prism may slow myopia progression and/or treat or prevent myopia.

The skilled person will understand that in an “oblique prism” the prism axis is not aligned with the horizontal (0-180 degree), or vertical (90 to 270 degree) meridian of the ophthalmic lens. As used herein “oblique” comprises predominantly vertically oriented oblique prisms and additionally comprises angles relative to the vertical axis up to 45 degrees.

As used herein, disease or condition associated with myopia includes any disease or condition that may result from or be associated with myopia. Examples of such disease and conditions include cataract, glaucoma, chorioretinal disease, retinal detachment, lacquer crack, chorioretinal atrophy, lattice degeneration and posterior vitreous detachment.

In the manner the present invention has great advantage in reducing the socio-economic cost of myopia and these associated diseases and conditions.

Further, the ophthalmic lens and/or an optical device comprising the optical lens according to the invention may advantageously slow progression of axial elongation of the eye to thereby control myopic progression and/or treat or prevent myopia or a disease or condition associated with myopia.

In a preferred embodiment the optical device of the invention comprises spectacles. As will be appreciated by the skilled person, the spectacles may comprise a right eye lens prescribed for the right eye and a left eye lens prescribed for the left eye.

Significantly, the invention is not limited to conventional spectacles and may also find application in variable lens technology. In a variable lens the optical components may be varied and selected in response to a change in gaze angle. In the current invention for example, the one or more oblique prismatic components or the central base-down prism in a distance zone may be altered with changing gaze angle.

The change may be implemented by for example, liquid crystal optics. The change in gaze angle may be tracked by a camera such as a rear ward facing micro-camera.

In one embodiment, the present invention relates to an ophthalmic lens comprising one or more oblique or vertical prismatic components.

Although not limited thereto, in one embodiment the one or more oblique or vertical prismatic components may be comprised in a distance zone of the ophthalmic lens. Based on the teaching herein the skilled person readily understands where the distance zone is located in an ophthalmic lens, which may be aligned with the centre of the pupil of the wearer during primary distance gaze.

In another embodiment the one or more oblique or vertical prismatic components may be comprised in a near zone of the ophthalmic lens.

In still another embodiment the one or more oblique or vertical prismatic components may be comprised in a distance zone and a near zone of the ophthalmic lens.

The lens of the invention may comprise one or more oblique or vertical prismatic components. The one or more vertical prismatic components may be (i) base-down. The one or more oblique prismatic components may be (ii) base down and base-in or (iii) base-up and-base-in.

Although not limited thereto, the one or more oblique or vertical prismatic components may be located centrally in the lens.

The skilled person understands that in one embodiment central means central to the direction of the gaze.

The skilled person understands that where more than one oblique or vertical prismatic components are involved they are in a central region or centrally positioned or oriented with respect to the line of sight. According to the invention the one or more oblique prismatic components may comprise 1 to 45° obliquity with respect to the vertical meridian (90°). In preferred embodiments the obliquity may be 5 to 45°, 5 to 35°, 5 to 25° or 5 to 10° with respect to the vertical meridian (90°). The obliquity may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45° with respect to the vertical meridian (90°).

The angle of the obliquity may be constant or variable. By variable is meant one location on the ophthalmic lens may comprise a different angle of obliquity of the prism to another location on the ophthalmic lens.

The magnitude of the one or more prismatic components may be constant or variable. Similarly to the above, by variable is meant one location on the ophthalmic lens may comprise a different magnitude of prism to another location on the ophthalmic lens.

The obliquity and/or power may increase from a distance centre to a near zone centre of the lens.

The obliquity and/or power may each be an independent continuous function.

In an illustrative embodiment the prism dioptric level and obliquity may increase from 1.0Δ and 0°, respectively; to 2.0Δ and 4°, respectively; to 3.0 A and 9°, respectively; to 4.0Δ and 21°.

In another illustrative embodiment comprising a plurality of base-down and base-in oblique prisms, the oblique prisms may comprise progressive addition of prismatic power from 4Δ to 8Δ.

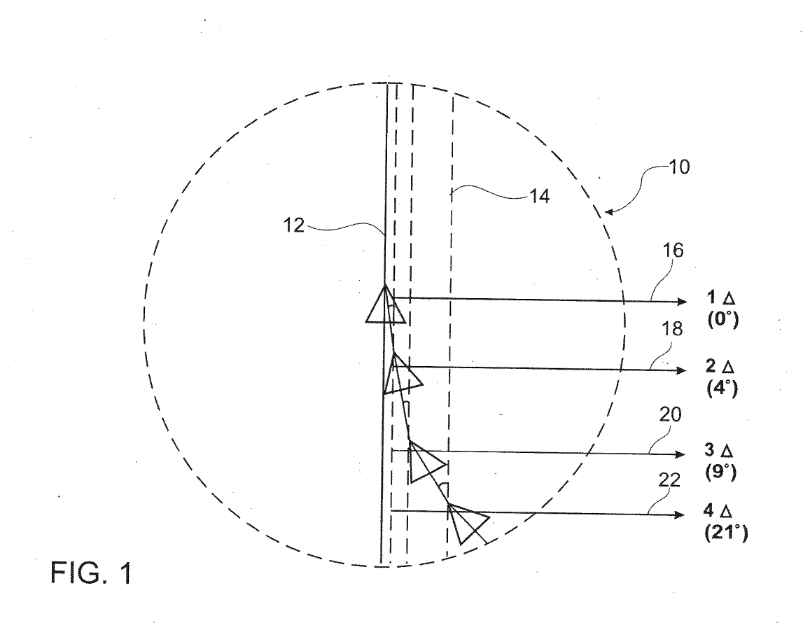

FIG. 1 shows an example of variable obliquity of the prism and variable power of the prism. In. FIG. 1 the variable obliquity and variable power increases from a distance centre 12 to near zone centre 14 of lens 10.

FIG. 1 also shows the variable obliquity and variable power to each be an independent continuous function. At zone 16 the prism dioptric level is 1.0Δ and the obliquity is 0°. At zone 18 the prism dioptric level is 2.0Δ and the obliquity is 4°. At zone 20 the prism dioptric level is 3.0Δ and the obliquity is 9°. At zone 22 the prism dioptric level is 4.0Δ and the obliquity is 21°.

The magnitude of the prism dioptric level of the one or more oblique prismatic components may be 0.1, 0.2, 0.3, 0.4, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 110.0, 11.5, 12.0, 12.5, 13.0, 13.5, 14.0, 14.5, 15.0, 15.5, 16.0, 16.5, 17.0, 17.5, 18.0, 18.5, 19.0, 19.5, or 20.0 prism dioptres. In other embodiments the dioptric power may be between 0.1 and 20.0, 0.5 and 15 or between 4 and 12 prism dioptres.

According to this invention the inter-pupillary distance may be 40 to 80 mm. In a preferred embodiment the inter-pupillary distance may be 55 to 70 mm. The inter-pupillary distance may be 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 or 80 mm.

The near inter-pupillary distance may be 35 to 75 mm. In a preferred embodiment the near inter-pupillary distance may be 48 to 68 mm. The near inter-pupillary distance may be 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74 or 75 mm.

The lens may further comprise progressive addition of positive dioptric power or one or more distinct zones of positive addition dioptric power. The positive dioptric power may comprise a single vision near power lens, a bifocal lens, a trifocal lens or a progressive style lens. In one embodiment the progressive or zonal dioptric power comprises a bifocal lens comprising the oblique prism in the lower reading segment.

According to the invention the slowing of myopia progression and/or treatment or prevention of myopia or a disease or condition associated with myopia may be over a period of time. The period of time may be weeks, months or years.

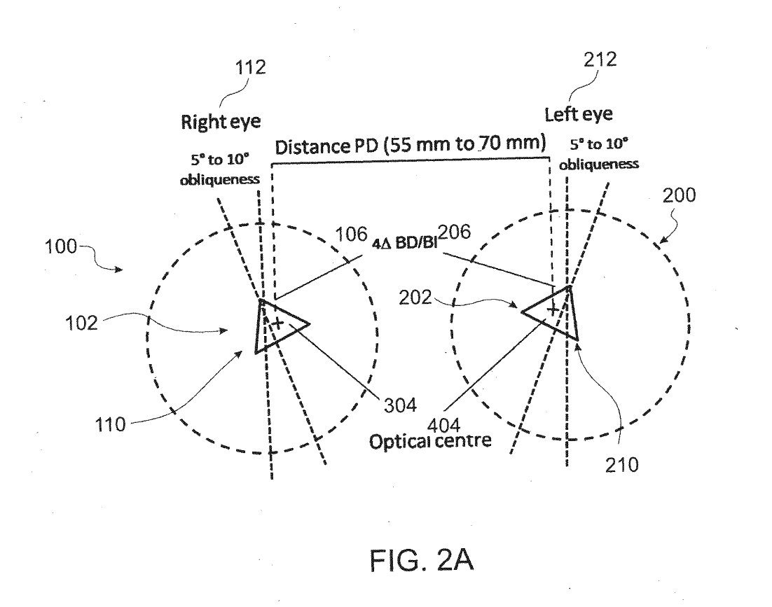

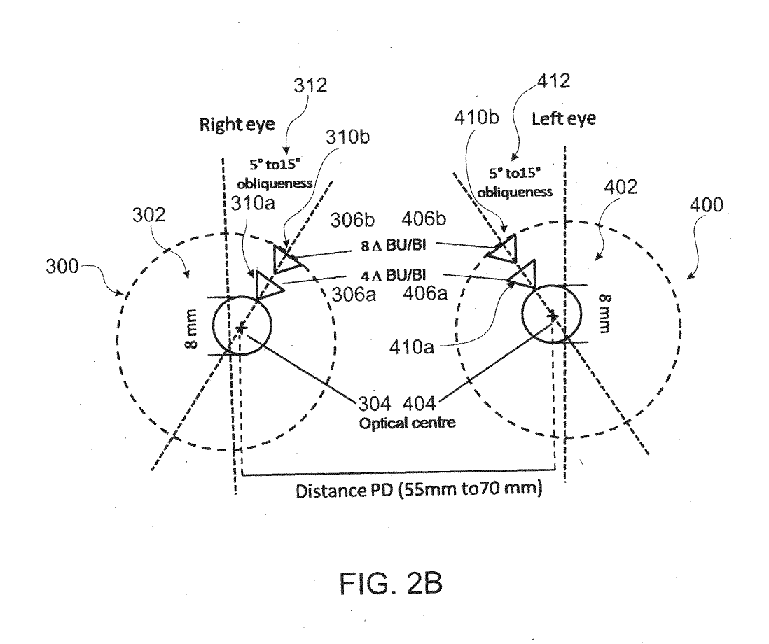

FIG. 2A shows one embodiment of the invention comprising right eye lens 100 and left eye lens 200. Lenses 100, 200 comprise a central oblique prism 110, 210 comprised of base-down and base-in located in a distance zone 102, 202.

In the embodiment of a lens 100, 200 according to the invention shown in FIG. 2A the prism dioptre 106, 206 comprises 4Δ. The skilled person is readily able to select an appropriate dioptric level to fulfill a prescription.

In another embodiment the central oblique prism may comprise a plurality of base-up and base-in oblique prismatic components.

As shown in FIG. 2A, the prism 110, 210 is positioned to include the optical centre 104, 204.

While the obliquity 112, 212 of lens 110, 210 is shown in FIG. 2A to be between 5° and 10° with respect to the vertical meridian (90°), based on the teaching herein a skilled person is readily able to select an appropriate angle.

FIG. 2B shows another embodiment of the invention comprising lenses 300, 400 comprising a plurality of base-up and base-in oblique prisms 310a, 410a, 310b, 410b also comprising progressive addition of positive dioptric power from 4Δ 306a, 406a, to 8Δ 306b, 406b.

While the obliquity 312, 412 is shown in FIG. 2B to be between 5° and 15° with respect to the vertical meridian (90°), based on the teaching herein a skilled person is readily able to select an appropriate angle.

The skilled person will readily understand that the embodiment shown in FIG. 2B is most suited to a single vision glass rather than a progressive one.

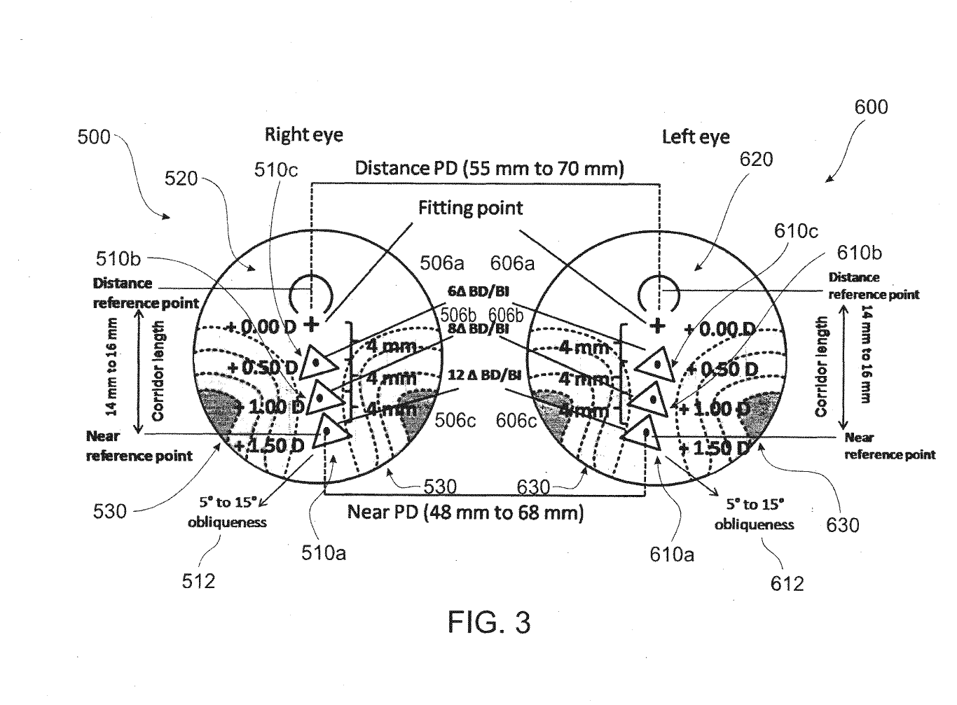

FIG. 3 shows another embodiment of the invention comprising lenses 500, 600. Lenses 500, 600 comprise variable oblique prisms 510a, 610a, 510b, 610b, 510c, 610c comprising variable progressive addition.

FIG. 3 shows lenses 500, 600 comprising a plurality of base-down and base-in oblique prisms 510a, 610a, 510b, 610b, 510c, 610c also comprising progressive addition of positive dioptric power from 6Δ 506a, 606a, to 8Δ 506b, 606b, to 12Δ 505c, 606c.

While the obliquity 512, 612 is shown in FIG. 3 to be between 5° and 15° with respect to the vertical meridian (90°), based on the teaching herein a skilled person is readily able to select an appropriate angle.

The non-shaded areas 520, 620 of the lens 500, 600 represent the optical zone/region of the lens, whereas the shaded regions 530, 630 typically contain unwanted optical distortions due to the design nature of progressive addition power designs.

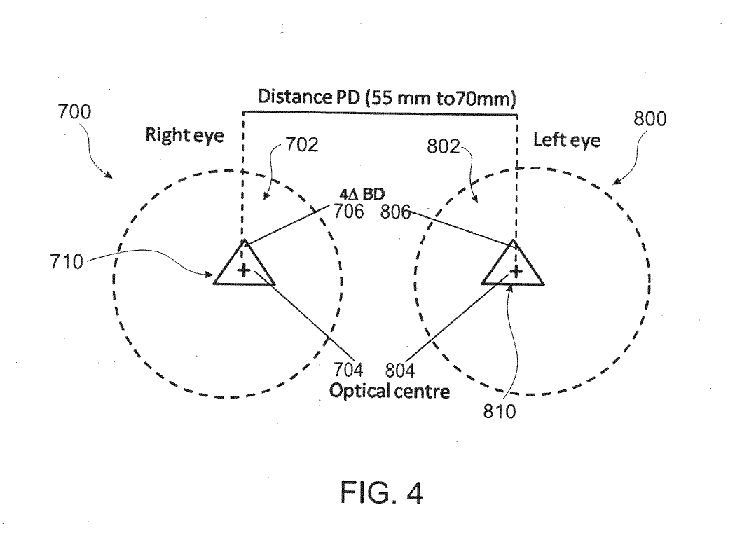

As shown in FIG. 4, the invention also provides an ophthalmic lens 700, 800 comprising a central base-down prism 710, 810 in a distance zone 702, 802 wherein the lens slows myopia progression and/or treats myopia.

The invention also provides an optical device comprising one or more ophthalmic lens 100, 200, 300, 400, 500, 600, 700, 800.

In a preferred embodiment of the first aspect, the optical device comprises spectacles.

According to the invention the slowing of myopia progression and/or treatment or prevention of myopia may comprise slowing or stopping an increase in axial length and/or reducing extra-ocular muscle tension or forces.

The invention also provides a method of slowing myopia progression and/or treating or preventing myopia including using one or more ophthalmic lenses according to the first or third aspect or an optical device according to the second or fourth aspect to thereby control myopia progression and/or treating or preventing myopia.

The invention further provides a method for slowing myopia progression and/or treating or preventing myopia including identifying optical or eye length changes associated with extra-ocular tension or muscle forces and correcting or inhibiting the optical or eye length changes with an ophthalmic lens according to the first or third embodiment or the optical device according to the second or fourth embodiment.

In one embodiment of the sixth aspect the optical or eye length changes may comprise changes due to extra-ocular muscle tension or muscle forces related to angled gaze.

The angles of gaze may be up and in or down and in.

The invention also provides a method for measuring a change in optical correction required with gaze angle. In one step this method comprises measuring a first axial measurement of an eye at a first gaze angle.

This method may also comprise the step of measuring two or more further axial measurements of the eye at respective additional and different gaze angles.

In another step this method may also comprise the step of analysing the first and further axial measurements to determine the change in optical correction of the eye required.

This method may further comprise applying the determined change in optical correction to design a lens.

According to the present invention also provided is an ophthalmic lens designed according to the method for measuring a change in optical correction required with gaze angle described above.

The invention provides a pair of spectacles comprising one or two ophthalmic lenses of the eighth aspect.

The one or two ophthalmic lenses may comprise a variable lens. The spectacles may be for intermediate and/or near tasks. The intermediate task may be for example, computer work. The near task may be for example, reading handheld material such as a book, c-book, tablet computer, phone, electronic device, magazine or newspaper.

The present inventors have also provided a method of designing a lens including measuring changes in axial length associated with gaze angle and eliminating or minimising the changes in axial length with a lens comprising one or more oblique prismatic components.

The measuring changes in axial length according to the above method of designing a lens may comprise the method for measuring a change in optical correction required as a result of gaze angle described above.

Importantly, the inventors have also provided a device for measuring an induced change in axial length with gaze angle. The device may comprise a biometer for measuring a first axial measurement of an eye at a first gaze angle and for measuring two or more further axial measurements of the eye at respective additional and different gaze angles.

The device may further comprise a tilting device for altering the angle of the biometer relative to the eye. The tilting device may comprise a mechanical device or an optical device. The device may comprise a head tracker to track a subjects' head position during the first and further measurements.

The head tracker may track head position in 3-axes comprising roll, pitch and yaw.

Designing a spectacle with oblique prism (base-down/base-in) that reduces extra-ocular muscles tension during reading or a near task, thus may lead to a decrease in myopia progression associated with near tasks or reading in children and young adults.

The present inventors investigated the changes in eye length and anterior biometrics occurring during 15° shifts in gaze direction over 5 mins. The inventors also investigated the relative effects of gravity and extra-ocular muscles on biometric parameter changes at 15° and 25° downward gaze over 5 mins.

The results provided herein show that the extra-ocular muscles appear to significantly elongate the eye during a short period of gaze at certain angles. Significantly, the inventors' have shown that subject with moderate levels of myopia had a greater axial elongation than subject with low levels of myopia and emmetropes at infero-nasal gaze, suggesting a correlation between the level of myopia and gaze angle associated axial elongation.

The following non-limiting examples illustrate the lenses, devices and methods of the invention. These examples should not be construed as limiting: the examples are included for the purposes of illustration only. The lenses, devices and method discussed in the Examples will be understood to represent an exemplification of the present invention.

### EXAMPLES

### Example 1

### Changes in Eye Length and Anterior Biometrics with Angular Shift in Gaze Direction and the Effects of Gravity and Extra-Ocular Muscles

The inventors investigated the changes in eye length and anterior biometrics occurring during 15° shifts in gaze direction over 5 mins as well as the relative effects of gravity and extra-ocular muscles on biometric parameters changes at 15° and 25° downward gaze over 5 mins.

### Materials and Methods

Thirty young subjects (10 emmetropes, 10 low myopes and 10 moderate myopes) were recruited in this study. Axial length measurements were taken from each subject's left eye through a rotating prism, along the foveal axis, using the Lenstar LS900 optical biometer at 0 and 5 mins, in nine cardinal gaze directions, with 15° deviation.

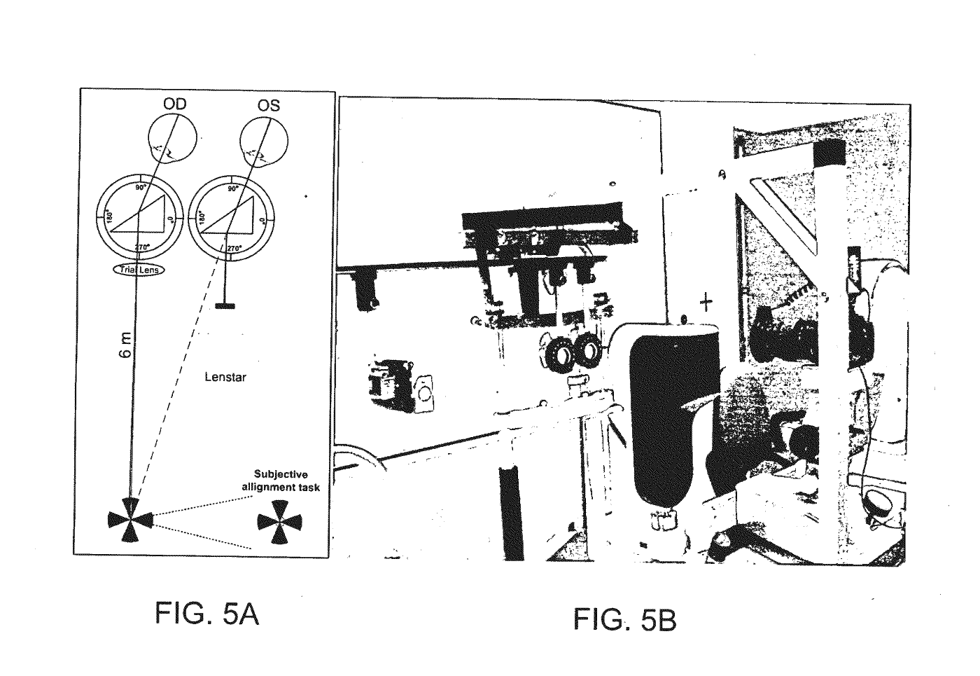

For all gaze conditions, the subject fixated on an external distance target with their optimally corrected fellow eye. Therefore in dichoptic view, an image of the external target was visible from the right eye and an image of the Lenstar's target was simultaneously seen from the left (tested) eye (FIGS. 5A and 5B).

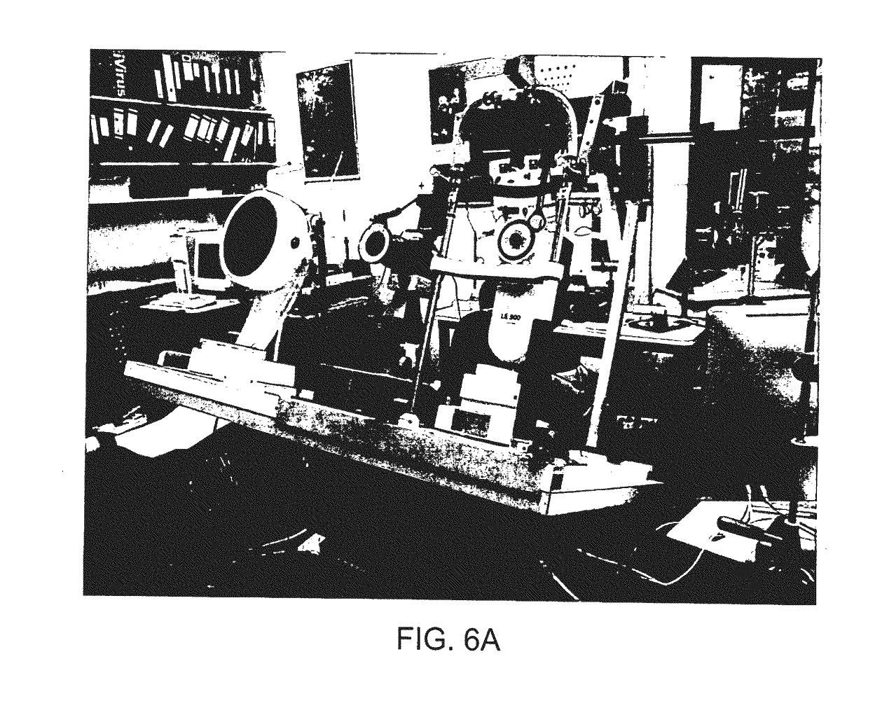

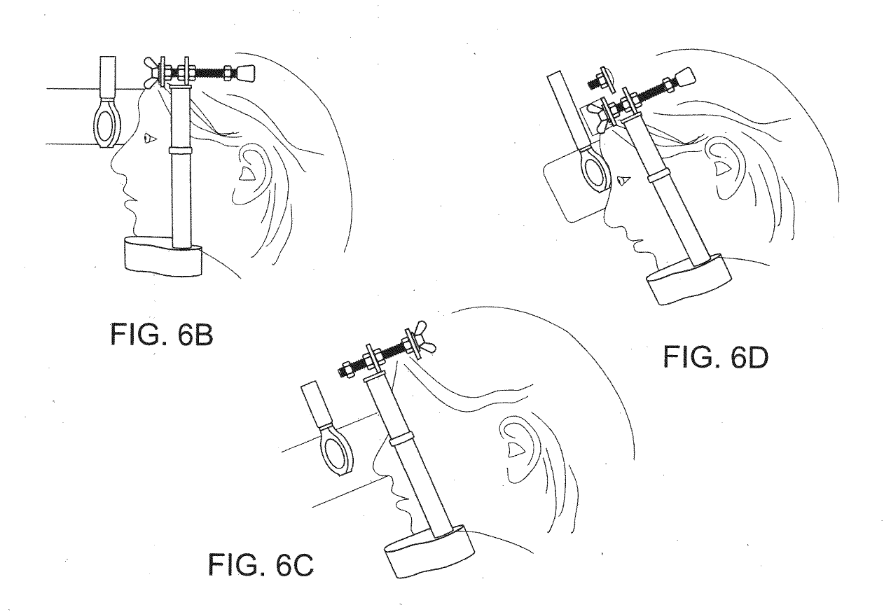

To further study the relative influence of gravitational force and extra-ocular muscles on the axial length changes in downward gaze, the Lenstar was inclined at 15° and 25° on a tilting table (FIG. 6A). Measurements of axial length were again performed over 5 mins under control condition with no head angle or eye tilt (FIG. 6B) and with two test conditions: i) looking down with head tilt but no eye turn (FIG. 6D); and ii) looking down without head tilt and with eye turn alone (FIG. 6C).

### Results

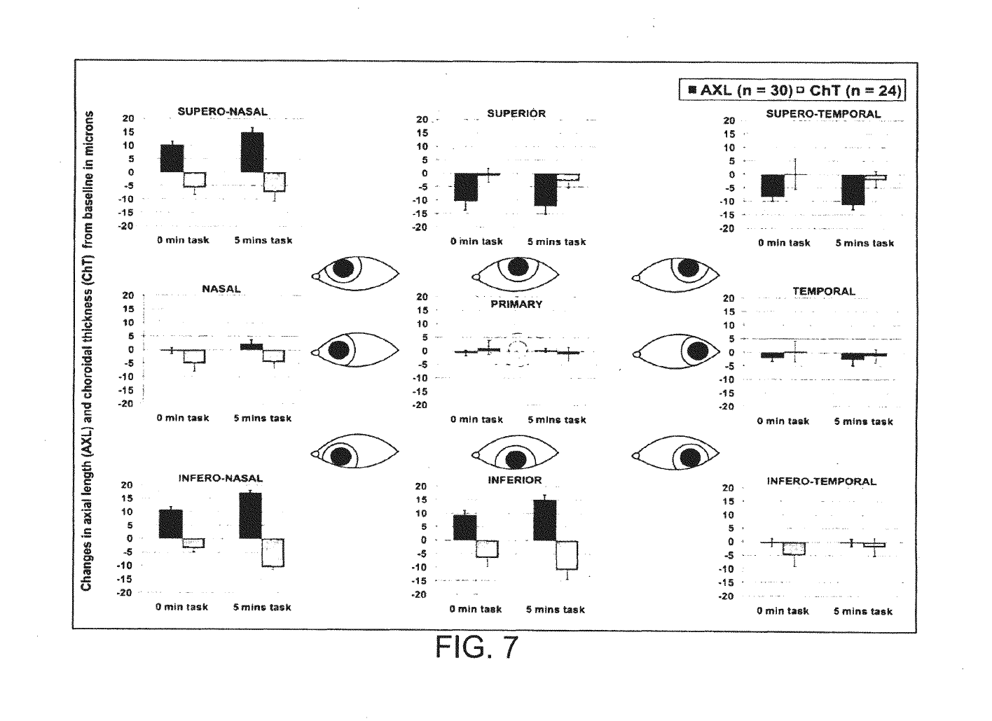

The results are summarised in FIG. 7 which shows the group mean changes of axial length (AXL) (n=30) in left eye (OS) in nine cardinal gaze directions with respect to baseline for the no accommodation condition over the 5 mins duration. Prior to each gaze measurements, the baseline value was taken after 10 mins of viewing a 6 meters target [i.e., with no accommodation (0 D)] in primary gaze.

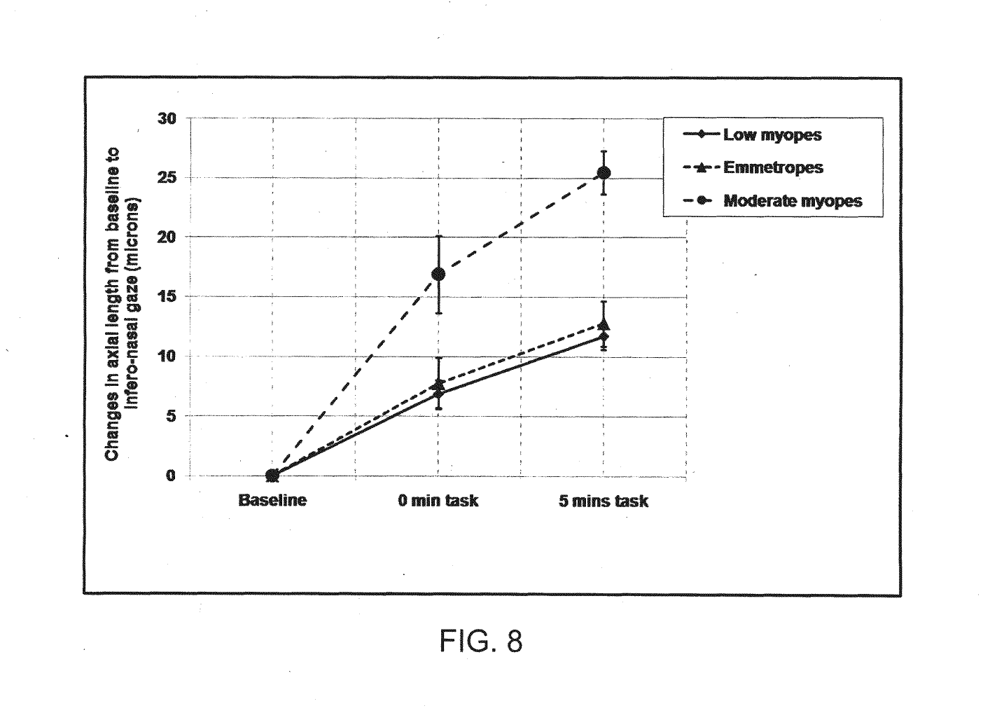

FIG. 8 shows a line graph summarising measured changes in axial length in the left eye (OS) in infero-nasal gaze after 5 mins of task with respect to baseline with no accommodation grouped by the subject's spherical refractive error. The baseline value was taken after 5 mins of viewing a 6 meters target [i.e., with no accommodation (0 D)] in primary gaze.

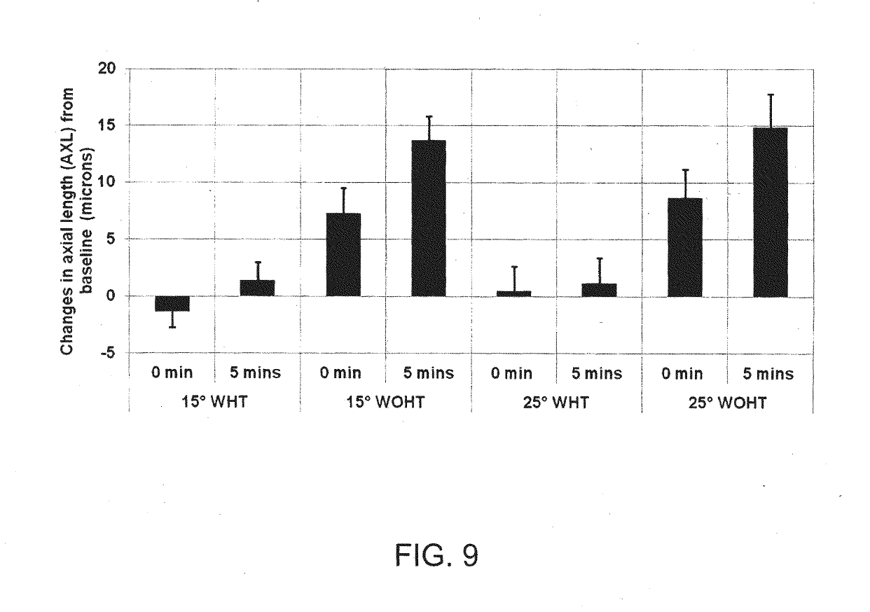

FIG. 9 shows a bar graph showing group mean changes of axial length (AXL) in left eye in 15° and 25° downward gaze with head tilt (WHT) (i.e., head-down and eyes straight) and without head tilt (WOHT) (i.e., head straight and eyes rotate down) with respect to baseline with the no accommodation condition over the 5 mins task. Prior to each test condition, the baseline value was taken after 5 mins of viewing a 6 meters target [i.e., with no accommodation (0 D)] in primary gaze. In this plot, AXL changed significantly in both 15° and 25° downward gaze directions without head tilt (ANOVA, p<0.05). However, AXL shows no significant changes in downward gaze with head tilt (both 15° and 25°) (ANOVA, p>0.05).

### Conclusions

The inventors have surprisingly shown the angle of gaze has a significant short-term effect on eye length, with greatest eye elongation occurring in the inferior nasal direction (such as typically occurs during reading).

The inventors have also surprisingly shown that subjects with moderate levels of myopia had a greater axial elongation than subject with low levels of myopia and emmetropes at infero-nasal gaze.

While not wanting to be bound by any one theory, the inventors propose the elongation of the eye appears to be due to the influence of the extra-ocular muscles, since the effect was eliminated when head turn was used instead of eye turn.

### Example 2

### Changes in Ocular Biometrics During Natural Visual Tasks

The inventors undertook further experiments to investigate the changes in axial length and ocular biometrics with a combined effect of accommodation and angle of gaze (convergence and downward gaze) during natural visual tasks (e.g. watching TV, computer work and reading books) over 5 mins in myopes and emmetropes.

### Materials and Methods:

Subjects:

Seven subjects participated in this study. Ages ranged from 18-29 years. There was no history of significant eye disease or injury. All subjects had best corrected visual acuity 20/20 or better in both eyes and normal levels of accommodation for the subject's age

### Instrumentation:

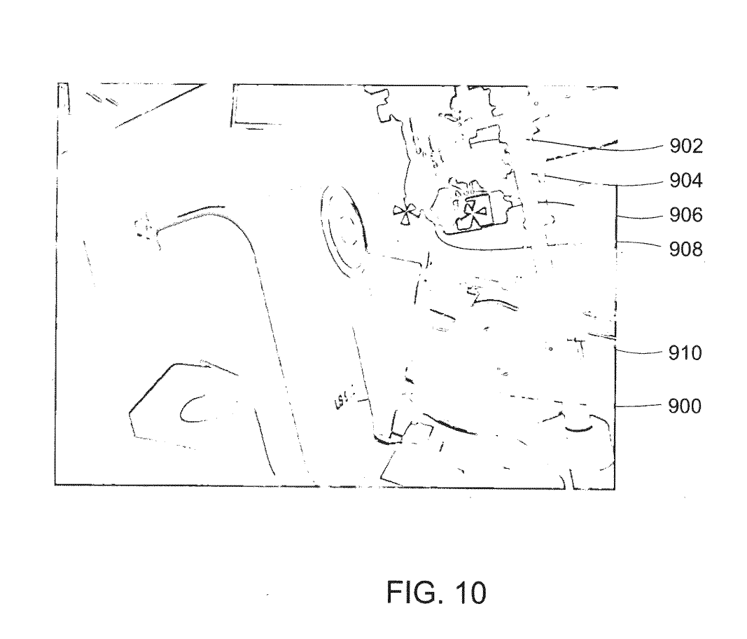

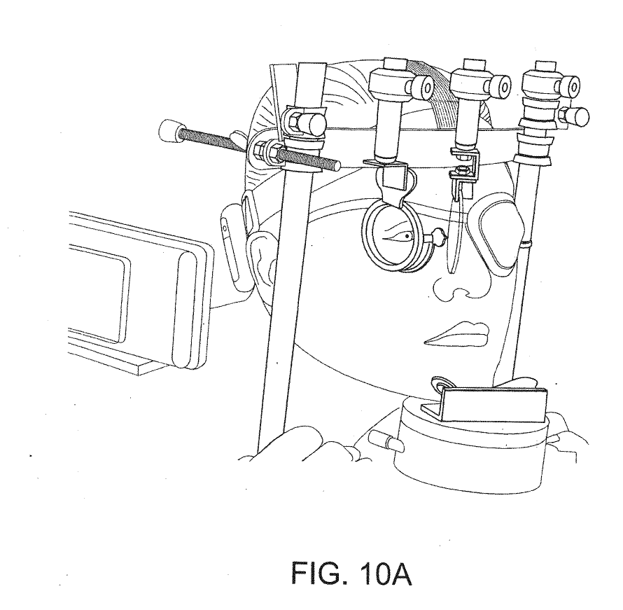

FIG. 10 shows a Lenstar LS900 900 (Haag-Streit international) comprising an adjustable head bar 902, a first surface mirror 904, a near target 906, a beam splitter 908 and a convergence control rotary stage 910. The Lenstar LS900 900 was shifted from its own commercial set-up, to a custom built height and tilt adjustable stage. The subject's chinrest was mounted on a rotary stage in such a way that various levels of convergence could be induced with rotation of the subject's head axis in primary or downward gaze.

FIG. 10A shows a subject performing a near task with 25° convergence and 2D accommodation in 10° downward gaze.

### Head Tracker:

Subjects wore a custom built 3-axis head tracker throughout the experiment that monitored subjects' relative head movements (roll, pitch and yaw) during measurements.

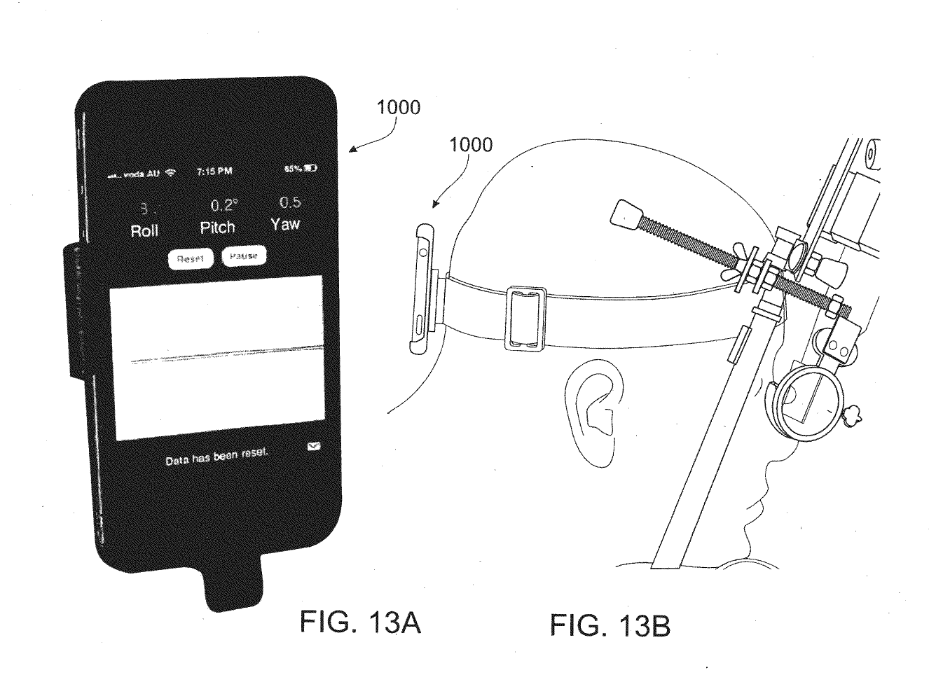

FIG. 13A (top) shows a front view of one embodiment of a head tracker 1000 according to the invention. FIG. 13B (bottom) shows the custom built head tracker 1000 shown in FIG. 13A mounted in use to a user's head.

### “Wash-Out Task”:

Initially, each subject performed a distance viewing task (subject's preferred task such as watching a DVD) binocularly at a 6 m distance.

### “Baseline Measurements”:

Axial length and ocular biometrics were then measured using the Lenstar LS900 in primary gaze at the end of the 5 mins “wash-out task”. These measurements were taken prior to each session.

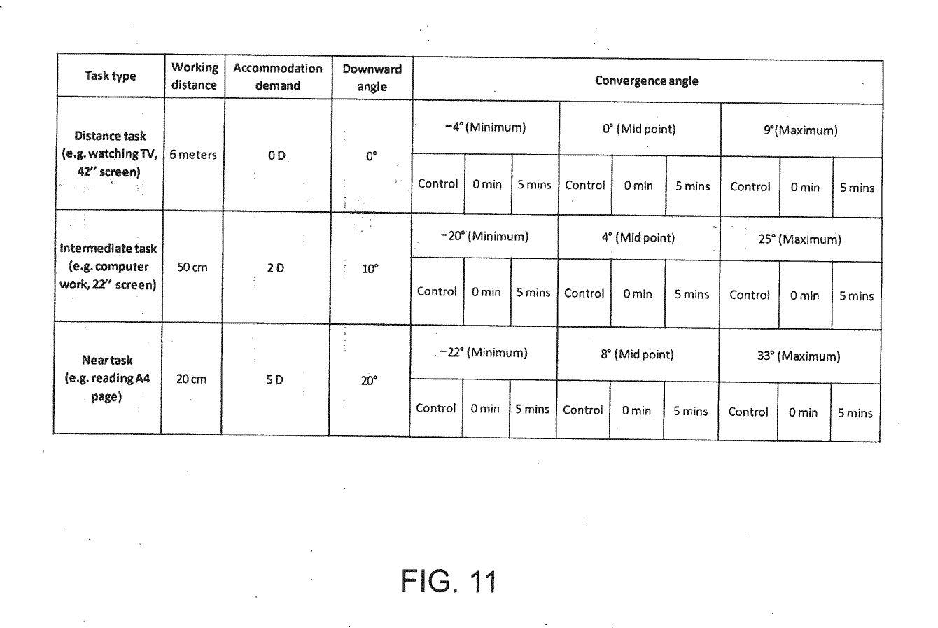

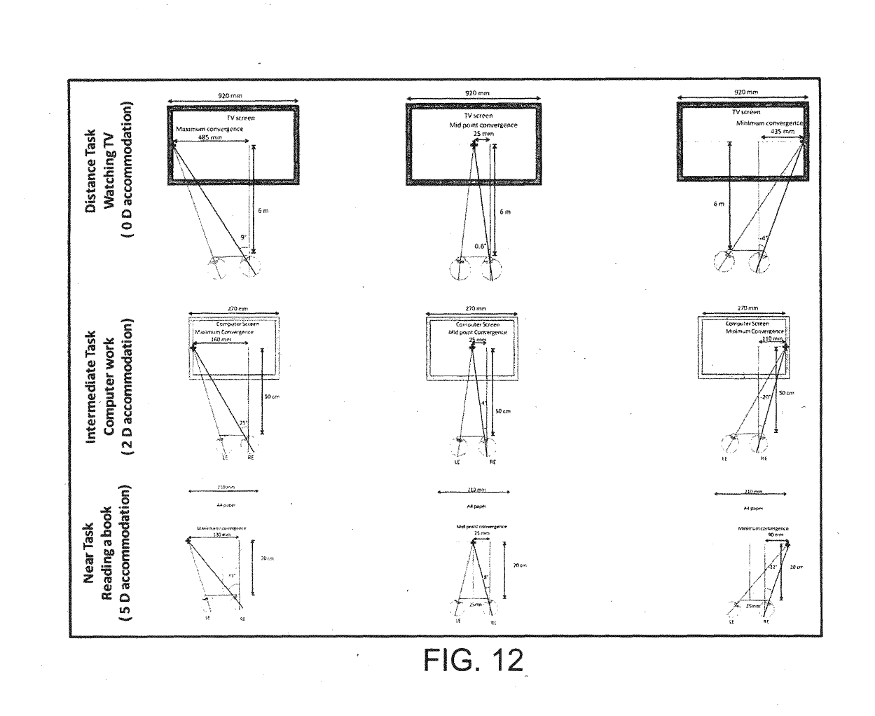

Subject's axial length and ocular biometrics were measured in nine different conditions of gaze over 5 mins time as illustrated in FIGS. 11 and 12.

Distance task (i.e. watching TV) corresponds to 0 D accommodation and 0° downward gaze (i.e. primary gaze), intermediate task (i.e. computer work) corresponds to 2 D accommodation and 10° downward gaze and a near task (i.e. reading books) corresponds to 5 D accommodation and 20° downward gaze.

These nine sessions for all gaze measurements (i.e. 3 levels of accommodation×3 levels of convergence) were completed across three days of testing (one accommodation condition on each day).

### Results:

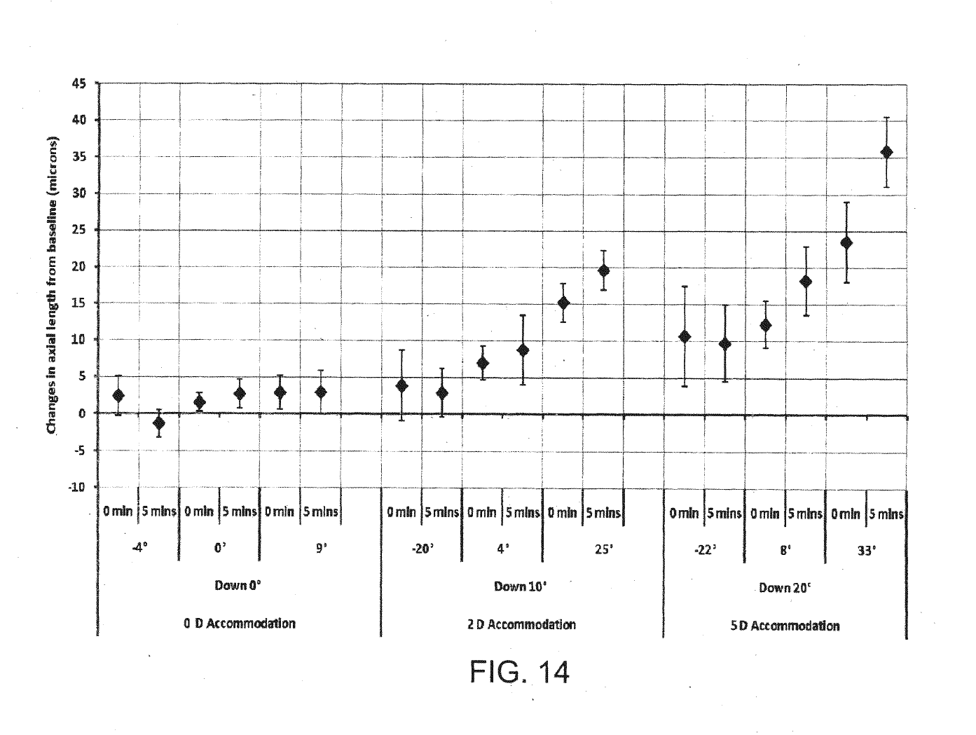

The highest change in axial length (group mean˜35 microns) took place after a 5 min of near task with 5 D accommodative demand, at 33° convergence in 20° downward gaze (see FIG. 14).

FIG. 14 illustrates the group mean changes of axial length (AXL) (n=7) in right eye during various natural visual tasks (i.e. watching TV, computer work and reading books) with a combined effect of accommodation, convergence and gaze angle. Positive angles correspond to convergence and negative angles correspond to divergence.

Axial length can also be increased moderately during intermediate distance tasks (e.g. computer work), particularly with convergence in downward gaze. However, there might be a substantially greater increase in axial length during close working distance tasks with convergence in downward gaze (e.g. reading books).

Distance tasks (e.g. Watching TV) may not cause a significant change in axial length.

From these results, it is apparent that changes in axial length of the eye during near work can be associated with the amount of accommodative demand, magnitude of convergence angle and magnitude of the downward angle.

### Summary:

Accommodation and down/inward gaze do cause small, short term increases in eye length.

The mechanism for these changes may be a combination of biomechanical forces and optical stimuli.

The cumulative effects of multiple short periods of near work activities employing downward gaze (e.g. reading in downward gaze, computer work or microscopy) over time could potentially promote a longer term elongation of the eye and the development of myopia through the mechanical influence of the extraocular muscles.

Throughout the specification the aim has been to describe the preferred embodiments of the invention without limiting the invention to any one embodiment or specific collection of features. It will therefore be appreciated by those skilled in the art that, in light of the instant disclosure, various modifications and changes can be made in the particular embodiments exemplified without departing from the scope of the present invention.

All computer programs, algorithms, patent and scientific literature referred to herein is incorporated herein by reference.

1. An ophthalmic lens comprising one or more vertical or oblique prismatic components, wherein the lens slows myopia progression and/or treats or prevents myopia or a disease or condition associated with myopia.

2. The lens of claim 1 wherein the one or each vertical prismatic component is a base down prism or the one or each oblique prismatic components is a base down and base-in prism.

3. The lens of claim 1 wherein the one or each oblique prismatic component is a base up and base-in prism.

4. The lens of claim 1 wherein the one or each oblique prismatic component is central to the direction of the gaze.

5. The lens of claim 1 wherein the one or more oblique prismatic in a distance zone and/or a near zone of the ophthalmic lens.

6. The lens of claim 1 wherein the one or more oblique prismatic components are oriented at 1 to 45° obliquity to the vertical meridian(90°).

7. (canceled)

8. (canceled)

9. (canceled)

10. (canceled)

11. The lens of claim 1 wherein an angle of the obliquity and/or a magnitude of the angle or dioptric power of the one or more oblique prismatic components comprises a continuous function or comprises variation from the distance centre to the near zone centre.

12. (canceled)

13. The lens of claim 1 wherein the lens further comprises progressive addition of positive dioptric power.

14. (canceled)

15. The lens of claim 1 wherein the one or each oblique prismatic component is a central base-down and base-in oblique prism.

16. (canceled)

17. (canceled)

18. The lens of claim 1 wherein the ophthalmic lens reduces extra-ocular muscle tension and/or other forces during activities that require angled gaze, wherein the angled gaze is down, down and in or up and in.

19. (canceled)

20. (canceled)

21. (canceled)

22. (canceled)

23. (canceled)

24. An optical device comprising one or more ophthalmic lenses, the one or more lenses each comprising one or more vertical or oblique prismatic components, wherein the lens slows myopia progression and/or treats or prevents myopia or a disease or condition associated with myopia.

25. (canceled)

26. (canceled)

27. A method of slowing myopia progression and/or treating or preventing myopia or a disease or condition associated with myopia including using one or more ophthalmic lenses to thereby slow myopia progression and/or treat or prevent myopia, the one or more lenses each comprising one or more vertical or oblique prismatic components, wherein the lens slows myopia progression and/or treats or prevents myopia or a disease or condition associated with myopia.

28. The method of claim 27 further comprising identifying optical or eye length changes associated with extra-ocular muscle tension or forces and correcting the optical or eye length changes using the one or more ophthalmic lenses.

29. The method of claim 28 wherein the optical changes comprise changes due to extra-ocular muscle tension and/or other forces related to angle of gaze, wherein the angled gaze is up and in, down, or down and in.

30. (canceled)

31. A method for measuring a change in optical correction required with gaze angle comprising:
measuring a first axial measurement of an eye at a first gaze angle;
measuring two or more further axial measurements of the eye at respective additional and different gaze angles; and
analysing the first and further axial measurements to determine the change in optical correction of the eye required.

32. The method of claim 31 further comprising applying the determined change in optical correction to design a lens.

33. (canceled)

34. (canceled)

35. (canceled)

36. (canceled)

37. A method of designing a lens including measuring changes in axial length associated with gaze angle and eliminating or minimising the changes with a lens comprising one or more oblique prismatic components.

38. The method of claim 37 wherein the step of measuring changes in axial length comprises the steps of:
measuring a first axial measurement of an eye at a first gaze angle;
measuring two or more further axial measurements of the eye at respective additional and different gaze angles; and
analysing the first and further axial measurements to determine the change in optical correction of the eye required.

39. A device for measuring a change in optical correction required with gaze angle comprising:
a biometer for measuring a first axial measurement of an eye at a first gaze angle and for measuring two or more further axial measurements of the eye at respective additional and different gaze angles.

40. The device of claim 39 further comprising a tilting device for altering the angle of the biometer relative to the eye and/or a head tracker to track a subject's head position during the first and further axial measurements.

41. (canceled)

42. (canceled)

43. (canceled)
